(19) United States
(12) Plant Patent Application Publication
(10) Pub. No.: US 2022/0386518 P1
(43) Pub. Date: Dec. 1, 2022

(54) Baptisia plant named 'Plum Rosy'

(50) ;

(71) Applicant: Hans A. Hansen, Zeeland, MI (US)

(72) Inventor: Hans A. Hansen, Zeeland, MI (US)

(73) Assignee: Walters Gardens Inc., Zeeland, MI (US)

(21) Appl. No.: 17/300,370

(22) Filed: May 28, 2021

(51) Int. Cl. A01H 6/50

(52) U.S. Cl. PLT/263.1

(57) ABSTRACT

A new and distinct plant cultivar of hardy herbaceous false indigo plant named Baptisia ‘Plum Rosy’ characterized by flowers that open up mauve-pinkish and lighten to a soft pink producing a bi-color spike colored on long spikes beginning in late May and continuing for two to three weeks. The new plant has a narrow, tall, upright, vase-shaped, multi-stemmed, winter-hardy habit with glaucous medium-green tri-foliate foliage and is suitable for landscaping as a specimen or en masse.

Botanical classification: Baptisia hybrid.

Cultivar designation: ‘Plum Rosy’.

### STATEMENT REGARDING PRIOR DISCLOSURES UNDER 37 CFR 1.77(b)(6)

Baptisia ‘Plum Rosy’ was promoted in a non-enabling descriptions and photographs on a website operated by Walters Gardens, Inc. on Feb. 1, 2020, followed by the “Walters Gardens 20-21 Catalog” distributed by Walters Gardens, Inc. on May 20, 2020. The first enabling disclosure of Baptisia ‘Plum Rosy’, in the form of a sale, was made by Walters Gardens, Inc. on Jun. 1, 2020 to Hopewell Nursery. Information and plants for this sale and all sales thereafter were obtained from the inventor. No plants of Baptisia ‘Plum Rosy’ have been sold, in this country or anywhere in the world, nor has any disclosure of the new plant been made, more than one year prior the filing date of this application, and such sale or disclosure within one year was either derived directly or indirectly from the inventor.

### BACKGROUND OF THE INVENTION

The present invention relates to a new and distinct cultivar of hybrid Baptisia plant, botanically known as Baptisia ‘Plum Rosy’ and will be referred to hereafter by its cultivar name, ‘Plum Rosy’, and the new plant. The new plant represents a new false indigo, a hardy herbaceous perennial grown for landscape and cut flower use.

‘Plum Rosy’ arose from an ongoing breeding program of the inventor at a nursery in Waseca, Minn. with continued evaluation at a wholesale perennial nursery in Zeeland, Mich. with the specific intention of improving garden worthiness of perennial false indigo plants with a wider variety of flower colors and improved garden habit.

Baptisia ‘Plum Rosy’ was a single seedling selection from a cross between a proprietary unreleased hybrid selection of Baptisia cinera times a proprietary unreleased hybrid selection of Baptisia minor (not patented) as the female or seed parent times a proprietary unreleased hybrid selection of Baptisia minor times a proprietary unreleased hybrid selection of Baptisia alba as the male or pollen parent. Seeds were collected from the individual selected female plant in fall of 2009 at the isolation block in Waseca, Minn., USA by the inventor. The seeds were sown by the inventor at a wholesale perennial nursery in Zeeland, Mich. in the fall of 2009 and the initial selection made in the spring of 2011 at the same nursery in Zeeland and given the breeder code H9-36-02.

‘Plum Rosy’ was initially asexually propagated by stem cuttings at a wholesale perennial nursery in Zeeland, Mich. in 2015. The resultant plants have demonstrated that the new plant has remained stable and true to type in successive generations of asexual propagation.

The nearest comparison plants known to the inventor are ‘Pink Truffles’ U.S. Plant Pat. No. 26,588, ‘Grape Taffy’ U.S. Plant Pat. No. 26,587, ‘Violet Dusk’ U.S. Plant Pat. No. 32,553, ‘Lavender Stardust’ (not patented) and ‘Pink Lemonade’ U.S. Plant Pat. No. 30,669. ‘Pink Truffles’ is taller and broader in habit with smaller flowers that are similar in color, the flower color is retained from anthesis to maturity, and the scapes are lighter gray-green. ‘Grape Taffy’ is much shorter in habit and the flowers are deep purple. ‘Violet Dusk’ is slightly taller in height and width and the flowers have a less distinct bi-color effect. ‘Lavender Stardust’ has a similar habit, but the flowers have a pale yellow keel to go with the dusty lavender banner. ‘Pink Lemonade’ has flowers that open lighter and turn dark while flowers of the new plant open darker and turn lighter.

Compared with typical Baptisia australis, the new plant has narrower and more upright habit and longer inflorescence. Compared with Baptisia minor the new plant is taller in habit and has a longer inflorescence. Typical Baptisia alba typically has white flowers. Typical Baptisia cinera has much shorter inflorescence of creamy-white flowers that face more sideways and less upwardly, and the habit is more compact. The female parent has lavender flowers on more compact plant and the inflorescence is arching to more horizontal and less upright. The male parent has similar habit, but the flowers are darker blue.

The new plant differs from all Baptisia known to the inventor in the following combined traits:
1. Flowers open up mauve pink and lighten before dropping to soft pink producing a bi-color spike;
2. Flowering begins early to late-May in Michigan and continues for about two to three weeks;
3. Narrow, tall, upright, vase-shaped, multi-stemmed, winter-hardy habit;
4. Glaucous, medium-green, tri-foliate foliage;

### BREF DESCRIPTION OF THE DRAWINGS

The photographs of the new plant demonstrate the unique traits of the new plant and the overall appearance. The colors are as accurate as reasonably possible with color reproductions. Variation in ambient light spectrum, source and direction may cause the appearance of minor variation in color. The accompanying photograph is of a eight-year-old plant growing in an open full-sun trial garden in Zeeland, Mich.

FIG. 1 shows the habit of a plant in full flower.

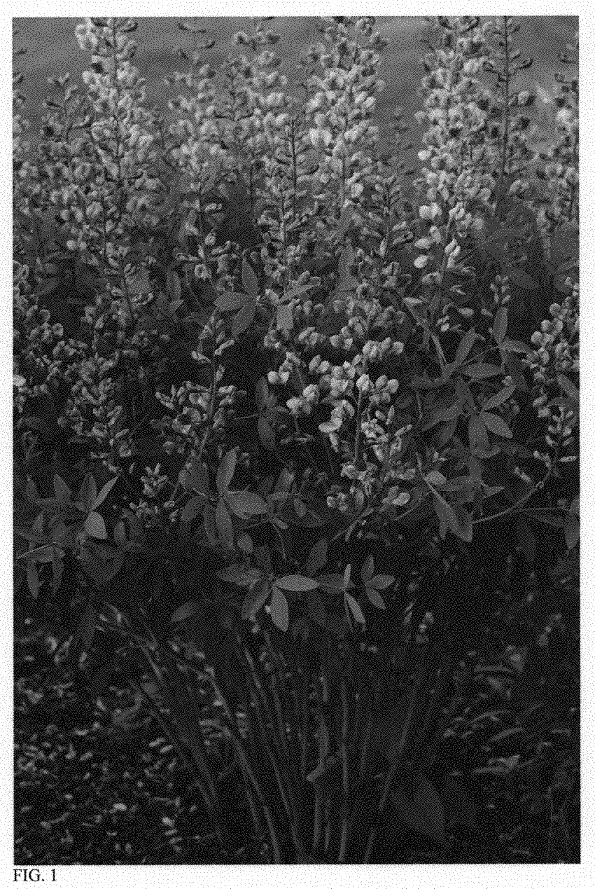

FIG. 2 shows a close-up of the inflorescence.

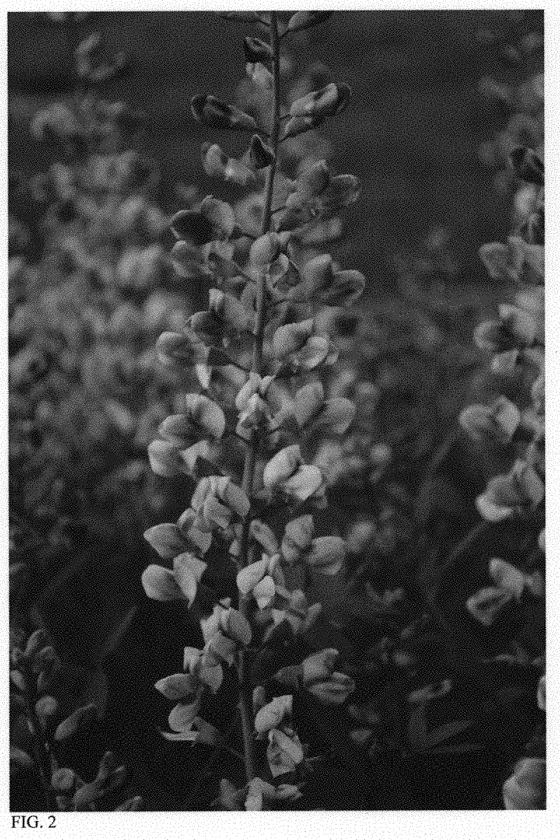

### DETAILED BOTANICAL DESCRIPTION OF THE PLANT

The following is a detailed description of a ten-year-old plant of ‘Plum Rosy’ as grown outdoors in a trial block at a wholesale perennial nursery in Zeeland, Mich. Plants of the new cultivar have not been tested under all possible conditions. The phenotype may vary with changes in environment, climate, and cultural conditions without change however in the genotype. The color references are in accordance with the 2015 edition of The Royal Horticultural Society Colour Chart except where general color dictionary terms are used.
Plant habit: Perennial, compact, well-branched, many-stemmed, vase-shaped growth habit with long inflorescences held above the foliage;
Plant size: Stems and crown about 32 cm across at soil level, at flowering about 72 cm tall from soil to top of flowers and 90 cm wide just below initial flowers; at maturity plant foliage height about 105 cm tall and about 110 cm wide;
Roots: Fibrous, well-branched, long, deeply rooted;
Root color: Nearest RHS 161D;
Propagation method: Stem cuttings, rooting in about three weeks;
Growth rate: Moderate to average;
Stems: Rigid and upright; highly glaucous; glabrous; cylindrical with shallow longitudinal furrows; lower two to three nodes without leaves or branches; normally two to three branches per plant below flowers; about 55 stems per clump;
Stem size: Main stem to about 8.0 mm diameter at base and 105 cm tall, average about 95 cm long and about 7.0 mm diameter; to about 43 cm long from soil to below initial flowers;
Stem branches: Primary branches at 45 to 55 degree angle above horizontal, up to 30.0 cm long and 5.0 mm diameter, average for primary branches about 26.0 cm long and 4.0 mm diameter, smaller distally; and two to four alternate secondary branches per stem of about 15 cm long and 3.0 man diameter, averaging about 12.0 cm long and 3.0 mm diameter;
Stem color: Between RHS 138B and RHS 148C;
Stem scales: Lanceolate; at stem nodes; dehiscing to leave behind thin scar on stem; about 18.0 mm wide at base and 23 mm wide in center; about 35.0 mm long and 2.0 mm wide in center of retuse apex with sharply pointed sides; truncate clasping base;
Stem scale color: Variable, nearest RHS N186D, RHS N186C and RHS 147D before dehiscing;
Internodes: Up to 12.0 cm apart between lowest branches, average about 8.5 cm;
Internode color: Between RHS 138D and RHS 148D with frequent marks of nearest RHS N186A;
Foliage: Alternate; ternate to palmately compound with three leaflets; outer two leaflets independent, at about 90 degree angle to middle leaflet; up to 7.0 cm long and 9.4 cm wide;
Leaflet: Three; obovate; apex acute, base cuneate; margins entire; petiolate; adaxial and abaxial surfaces matte, glabrous; slightly glaucous adaxial and glaucous abaxial; middle lobe to about 5.6 cm long and 3.0 cm wide, side lobes about 5.0 cm long and 2.8 cm wide;
Leaflet color: Newly expanding adaxial nearest RHS 146B and abaxial nearest RHS 147C; mature adaxial and abaxial nearest RHS 147B;
Venation: Pinnate, glabrous, thin, not conspicuous;
Vein color: Newly expanding foliage adaxial midrib nearest RHS 146B and abaxial nearest RHS 147C; mature midrib nearest RHS 138C above and lighter than RHS 145D below; secondary veins same color as surrounding leaf tissue;
Petioles: Glabrous; slightly glaucous; cylindrical; to about 9.0 mm long and 2.0 mm wide at base, average 7.0 cm long and 1.5 mm wide;
Petiole color: Adaxial nearest 146D, abaxial nearest RHS 147B;
Stipules: Narrowly lanceolate, acute apex with base truncate; to about 28.0 mm long and 8 mm wide, average 24.0 mm long and 6.0 mm wide with largest stipules below primary branches and decreasing distally and on secondary branches;
Stipule color: Nearest RHS 138A both surfaces;
Peduncle: Cylindrical with vertical shallow ridges and furrows; glaucous; glabrous; from first flower to apex about 29 cm long; diameter at the base of first flower about 3.5 mm and about 2 mm diameter at the apex;
Peduncle color: Nearest RHS 148C in proximal and nearest RHS N186C in distal flowering portions;
Pedicel: Cylindrical, glabrous, glaucous; about 10.0 mm long and 1.0 mm diameter;
Pedicel color: Dorsally nearest N186B, ventrally nearest RHS 146C;
Calyx: Campanulate; four-lobed; 12.0 mm long, 9.0 mm tall and 11.0 mm across at apex;
Sepals: Four; one larger dorsal, one ventral and two lower lateral; acute apices; fused into tube in proximal 9.0 mm; margin entire; glabrous adaxial and abaxial; dorsal sepal 12.0 mm long and 7.0 mm wide at fusion; other three sepals 12.0 mm long and 4.0 mm across at fusion;
Sepal color: Adaxial between RHS 144B and RHS 144A; abaxial nearest RHS 144A;
Buds one day prior to anthesis: Oblong elliptic, flatted vertically; about 25.0 mm long and 9.0 mm tall and 7.0 mm wide;
Bud color: One day prior to opening exposed keel petal blend between RHS 79D and RHS N80D, enfolded banner petal between RHS N8OD and RHS N77D, exposed alae petals nearest RHS 1D;
Flower: Zygomorphic, papilionaceous, non-secund, held at about 30 degree angle above horizontal; about 38 flowers per main raceme and about 16 per secondary branch; seasonally effective for about 2 to 3 weeks beginning in early to mid-June in Zeeland, Mich.; individual flower remain effective and on raceme for about four days; individually about 26.0 mm long, 17.0 mm tall and 18.0 mm wide at largest portions; consisting of an upper banner, a lower keel made up of two lobes folded around gynoecium and androecium; and two lateral wings or alae laterally appressed against keel;
Flower fragrance: None detected;
Petals: Five; with a lower fused keel, an upper banner, and two lateral wings or alae; keel comprised of two sections that are folded around stamens and pistil;
   Banner petal.—Conduplicate, curved upward and backward and pinched in the middle; apex retuse, base claw-like, margin entire; about 21.0 mm long, 12.0 mm across natural width, 21.0 mm wide flattened spread and 8.0 mm tall; with rounded emarginate apex notched about 4.0 mm deep; basal portion attenuate claw-type, 5.0 mm long and 3.0 mm wide.
   Banner color.—Adaxial between RHS N77D and RHS N80D with eye heavily maculate nearest RHS N77A surrounding a center 2.0 mm diameter of RHS 145B and claw base nearest RHS 145C; abaxial toward perimeter closer to RHS N80D than RHS N77D, center nearest RHS N77D with claw nearest RHS 145C.
   Keel.—Comprised of two main lobes that are folded around stamens and pistil; fused in the distal one-third with the apex emarginate or retuse and the bases separate and claw-like; margin entire; top edge about one-third of the way from base has 2.0 mm smaller lobe pointing toward base; about 23 mm long and 11.0 mm tall and 2.0 mm wide; blade portion to about 18.0 mm long and 10.0 mm tall, with claw to about 7.5 mm long and base narrowed to 2.0 mm wide for the proximal 3.0 mm.
   Keel color.—Adaxial base nearest RHS 145D, center nearest RHS 79D with margins nearest RHS N80D; abaxial keel margins lighter than RHS N80D and center lighter than RHS 79D, base nearest RHS 145D;
   Alae.—Two; papilionaceous corolla appendage with rounded apex and claw-like base; with 2.0 mm long by 2.0 mm wide lobe pointing toward base and about one-third of the way from base; about 22.0 mm long and 10.0 mm tall with the claw to 3.0 mm wide at base of blade and 2.0 mm wide at base and 8.0 mm long; blade about 14.0 mm long and 10.0 mm wide;
   Alae color.—Adaxial claw base nearest RHS 145D; main blade portion and lobe nearest RHS 1D; abaxial claw base nearest RHS 145D, small lobe and main lobe portion nearest RHS 1D;
   Mature keel and upper banner.—Nearest RHS 84D.
Receptacle: Disk-shaped, about 4 mm diameter and 1.2 mm depth; color nearest RHS 138A;
Gynoecium: One, with superior ovary and stipe; to about 22.0 mm long;
   Style.—Cylindrical; glabrous; slightly glaucous; tapered and curved near stigma, about 9.0 mm long and about 0.7 mm diameter above ovary; color between RHS 154D and RHS N144D.
   Stigma.—About 0.2 mm diameter; color nearest RHS 150C.
   Ovary.—Superior above hypanthium, suspended by stipe; about 7 mm long and about 1.7 mm in diameter; color between RHS 154D and RHS N144D.
   Stipe.—Cylindrical; glabrous; slightly glaucous; about 5 mm long and 0.7 mm diameter; color between RHS 154D and RHS N144D.
Androecium:
   Stamens.—Ten.
   Filament.—Glabrous; slightly lustrous; not united 20.0 mm in length and about 0.5 mm in diameter; slightly curved upward distally; color nearest RHS 1D.
   Anther.—Dorsifixed, oblong; about 2.0 mm long and 1 mm wide; color nearest RHS 17A.
   Pollen.—Spherical; abundant; color nearest RHS 17A.
Fruit: Bivalve inflated ellipsoidal pod; glabrous; about 33.0 mm long, 20 mm across and 14 mm thick; with thin, linear, arcuate, terminal beak about 3.0 mm long and about 1 mm thick; rounded base;
Fruit color: At maturity between 200A and RHS 202A;
Seeds: About 8 per pod (open pollinated); reniform; glabrous; about 4.0 mm long, 3.0 mm across and 2.0 mm thick; color nearest RHS N199C;
Hardiness: To USDA zones 4 to 9; tolerant of heavy clay or light loamy-sand soils; able to withstand drought conditions once established;
Diseases: Susceptibility or resistance to diseases beyond that typically found in other false indigo plants has not been observed;

It is claimed:

1. A new and distinct cultivar of hardy False Indigo plant Baptisia plant named ‘Plum Rosy’ as herein described and illustrated.
